(19) United States
(12) Patent Application Publication
(10) Pub. No.: US 2014/0171914 A1
(43) Pub. Date: Jun. 19, 2014

(54) MEDICAL DEVICES WITH A LOCKING MEMBER

(71) Applicant: BOSTON SCIENTIFIC SCIMED, INC., Maple Grove, MN (US)

(72) Inventors: TODD ROWE, EXCELSIOR, MN (US); BENJAMIN P. GUNDALE, PLYMOUTH, MN (US); PATRICIA JOHNSON, CO. GALWAY (IE)

(73) Assignee: BOSTON SCIENTIFIC SCIMED, INC., MAPLE GROVE, MN (US)

(21) Appl. No.: 14/106,267

(22) Filed: Dec. 13, 2013

Related U.S. Application Data
(60) Provisional application No. 61/737,610, filed on Dec. 14, 2012.

(51) Int. Cl. A61M 25/09

(52) U.S. Cl. CPC A61M 25/09
USPC 604/510; 604/528

(57) ABSTRACT

Medical devices and methods for making and using medical devices are disclosed. An example medical may include a guide catheter. The guide catheter may include an elongate catheter shaft having a proximal end, a distal end, and a central lumen extending therebetween. An inflatable locking member may be disposed within the lumen. The locking member may be configured to longitudinally secure the position of a medical device extending through the lumen relative to the catheter shaft. The guide catheter may also include an inflation lumen in fluid communication with the inflatable locking member.

### CROSS-REFERENCE TO RELATED APPLICATIONS

This application claims priority under 35 U.S.C. §119 to U.S. Provisional Application Serial No. 61/737,610, filed Dec. 14, 2012, the entirety of which is incorporated herein by reference.

### TECHNICAL FIELD

The present disclosure pertains to medical devices and methods for making and using medical devices. More particularly, the present disclosure pertains to guide catheter with a locking member.

### BACKGROUND

A wide variety of intracorporeal medical devices have been developed for medical use, for example, intravascular use. Some of these devices include guidewires, catheters, and the like. These devices are manufactured by any one of a variety of different manufacturing methods and may be used according to any one of a variety of methods. Of the known medical devices and methods, each has certain advantages and disadvantages. There is an ongoing need to provide alternative medical devices as well as alternative methods for manufacturing and using medical devices.

### BRIEF SUMMARY

This disclosure provides design, material, manufacturing method, and use alternatives for medical devices. An example medical may include a guide catheter. The guide catheter may include an elongate catheter shaft having a proximal end, a distal end, and a central lumen extending therebetween. An inflatable locking member may be disposed within the lumen. The locking member may be configured to longitudinally secure the position of a medical device extending through the lumen relative to the catheter shaft. The guide catheter may also include an inflation lumen in fluid communication with the inflatable locking member.

Another example guide catheter may include an elongate catheter shaft having a central lumen defined therein. A balloon may be positioned within the lumen. The balloon may be configured to shift between a first configuration and an inflated configuration. The guide catheter may also include an inflation lumen that is in fluid communication with the inflatable locking member. When the balloon is in the inflated configuration, the balloon may longitudinally secure the position of a medical device extending through the lumen relative to the catheter shaft.

A method for longitudinally securing medical devices is also disclosed. The method may include disposing a first medical device within a body lumen. The first medical device may include an elongate shaft having central lumen defined therein, a balloon disposed within the lumen, and an inflation lumen in fluid communication with the balloon. The method may also include disposing a second medical device within the central lumen of the first medical device and inflating the balloon to longitudinally secure the position of the second medical device relative to the first medical device.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description in connection with the accompanying drawings, in which:

FIG. 1 is a partial cross sectional side view of an example medical device disposed within a blood vessel.

FIG. 2 is a partial cross sectional side view of an example medical device.

FIG. 2A is a partial cross sectional side view of an example medical device illustrated in FIG. 2 in a locked configuration.

FIG. 3 is a partial cross sectional side view of another example medical device.

FIG. 4 is a partial cross sectional side view of the example medical device illustrated in FIG. 3 in a locked configuration.

FIG. 5 is a cross-sectional view taken through line 5-5 in FIG. 4.

FIG. 6 is an alternative cross-sectional view of an example medical device.

FIG. 7 is an alternative cross-sectional view of an example medical device.

FIG. 8 is a partial cross sectional side view of another example medical device.

FIG. 9 is a partial cross sectional side view of another example medical device disposed within a blood vessel.

While the disclosure is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is noted that references in the specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used connection with other embodiments whether or not explicitly described unless clearly stated to the contrary.

The following detailed description should be read with reference to the drawings in which similar elements in different drawings are numbered the same. The drawings, which are not necessarily to scale, depict illustrative embodiments and are not intended to limit the scope of the invention.

For purposes of this disclosure, “proximal” refers to the end closer to the device operator during use, and “distal” refers to the end further from the device operator during use.

Embodiments of the disclosure may include a medical device such as a guide catheter having a catheter shaft, an inflatable locking member, and an inflation lumen. The inflatable locking member may include an inflatable balloon disposed within the lumen of the catheter shaft. The locking member may longitudinally secure the position of a medical device extending through the lumen relative to the catheter shaft by inflating the balloon. Upon inflation, the balloon may grip a medical device extending through the guide catheter and longitudinally secure the position of the medical device relative to the guide catheter.

Many of the following examples illustrate methods of using the guide catheter. For example, the guide catheter may be employed to assist stent delivery systems (SDS). It will be understood that this choice is merely exemplary and the guide catheter may be used in any desired body location requiring diagnostic or therapeutic modalities without departing from the scope of the present disclosure.

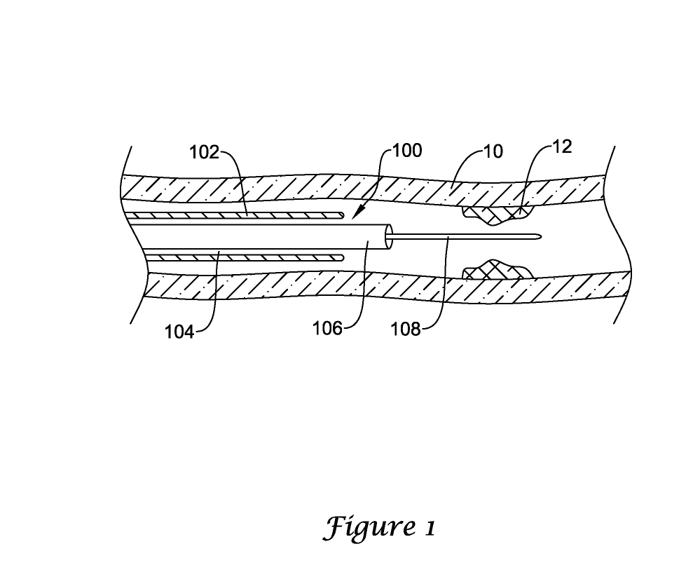

FIG. 1 is a schematic view illustrating an example medical device system 100. The figure depicts the system 100 within a blood vessel 10 of a patient. The system 100 may include a guide catheter 102 having a lumen 104 extending therethrough. Another medical device 106 may extend through the lumen 104 of the guide catheter 102. The “second” medical device 106 may be, for example, a stent delivery system, an angioplasty catheter, a dilation catheter, a cutting balloon catheter, a rotational atherectomy catheter, or the like. During use, the medical device 106 may be advanced through the guide catheter 102 and over a guidewire 108 to a position adjacent to an area of interest. When properly position, the medical device 106 may be used to perform a suitable diagnostic and/or treatment intervention.

The proper positioning of medical devices may contribute to the overall success of the intervention. For example, when delivering a stent, careful and accurate placement of the stent may correlate with the ultimate success of the procedure. In addition, maintenance of the position of one or more medical devices during the intervention (including stenting procedures, angioplasty, and/or medical procedure) may allow a clinician to efficiently perform the necessary parts of the intervention while minimizing additional placement and/or positioning steps that could prolong the procedure and/or increase the trauma to patient.

System 100 is designed to improve the positioning of medical devices so that medical interventions may be performed efficiently, with a relatively high level of accuracy, and with a reduction of trauma to the patient. For example, one or more structures of system 100 may include a locking member or positioning aid that helps to secure the relative longitudinal position of one medical device relative to another (e.g., the guide catheter 102 relative to the medical device 106. A variety of locking members are contemplated. Some of the locking members contemplated are disclosed herein and discussed in more detail below.

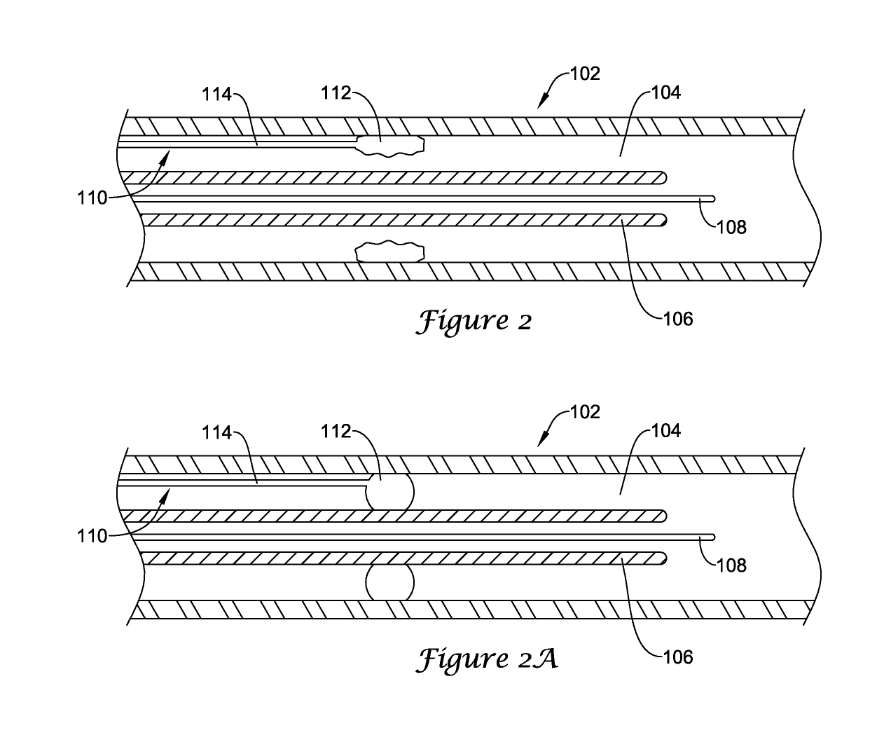

In at least some embodiments, the locking member(s) may be a feature of the guide catheter 102. For example, the guide catheter 102 may include a locking member 110 as shown in FIG. 2. In general, the locking member 110 may be configured to shift between a first or “unlocked” configuration and a second or “locked” configuration. In at least some embodiments, locking member 110 is an inflatable locking member that may include an inflatable balloon 112 and an inflation tube or lumen 114 in fluid communication with the balloon 112 (e.g., the inflation lumen 114 may be connected to a fluid source, such as a syringe pump, a mechanical or electrical pump, or the like). Other embodiments are contemplated included including “non-inflatable” locking member that may include an expandable frame or lock, a mechanical lock, or the like.

Locking member 110 may be used to reduce relative movement between the guide catheter 102 and medical device 106 or otherwise “lock” the position of medical device 106 as shown in FIG. 2A. For example, the balloon 112 may be inflated by passing inflation media through the inflation lumen 114 and into the balloon 112. The diameter of the inflation lumen 114 may depend upon the physical properties of the fluid in use and the amount of space left within the lumen 104 after inserting the medical device 106. When inflated, the balloon 112 may expand radially inward within the guide catheter 102 and contact or grip the medical device 106, thereby securing the position of the medical device 106 relative to the guide catheter 102. When deflated, the balloon 112 may generally conform to the inner surface of the guide catheter 102 so that medical devices (e.g., the medical device 106) may pass essentially freely therethrough.

In addition to securing the relative position of the guide catheter 102 and the medical device 106, the locking member 110 may provide additional desirable features. For example, the locking member 110 may be used to improve the pushability of medical device 106. For example, the medical device 106 may be designed to access relatively small vascular locations. Because of this, the medical device 106 may have a relatively small profile and/or be relatively highly flexible. This may pose challenges to advancing the medical device 106 through an occlusion or lesion.

Locking member 110 may be utilized to improve the pushability of medical device 106 and/or improve the ability of the medical device 106 to pass through occlusions/lesion. For example, the balloon 112 may be inflated to secure the guide catheter 102 to the medical device 106. This may be done when a relatively small portion of the length of the medical device 106 (e.g., on the order of about 5-20 cm, or about 10-12 cm or so) extends out from the distal end of the guide catheter 102. When so configured, the guide catheter 102 (which may be stiffer than the medical device 106) may be used to aid in pushing the medical device 106 (e.g., through an occlusion/lesion).

The form of the guide catheter 102 may vary. For example, the guide catheter 102 may have a suitable cross-sectional shape such as circular, oval, polygonal or the like. The guide catheter 102 may be dimensioned to enter relatively small vessels such as relatively small coronary arteries and/or vascular regions (e.g., the guide catheter 102 may have an 8F, 7F, 6F, 5F, or smaller diameter. The length of the guide catheter 102 may be chosen to accommodate the distance from the introduction point into the patient's vasculature to the planned location of stent deployment within the vasculature. The distal end of the guide catheter 102 may be chosen as an atraumatic device, designed to reduce inadvertent injury to the vascular tissue during advancement of the guide catheter 102. Additionally, the guide catheter 102 or a portion of it may be selectively steerable, employing mechanisms such as, pull wires, motors, hydraulics, or the like.

The inflatable locking member 110 may be any device or mechanism that may expand upon inflation to firmly grip the medical device 106, holding it in position. The inflatable locking member 110 may include one or more balloons, wire braids, baskets, or the like. This may include a plurality of balloons that are longitudinally spaced along the length of the guide catheter 102. In addition, the locking member 110 may be disposed a suitable position along the length of the guide catheter 102. This may include adjacent to the distal end of the guide catheter 102 or at essentially any other position.

In the inflated state, the inflatable balloon 112 may have a cylindrical, annular, semi-annular, polygonal, or any other shape. For example, in the inflated state, the inflatable balloon 112 may have an annular, doughnut-like, shape constricting the inner diameter of the lumen 104 of the guide catheter 102. The inflatable balloon 112 may have a thickness large enough to tightly fit within the lumen 104 along with the medical device 106. It may be sufficiently long to grip the medical device 106 firmly and reduce any relative motion between the medical device 106 and the guide catheter 102. The inflatable balloon 112 may be non-pliable or semi-pliable to increase the longitudinal force exerted upon the medical device 106. The balloon 112 may be formed from a variety of materials including those disclosed herein. This may include forming the balloon 112 from non-compliant balloon material such as PEBAX.

The inflation lumen 114 may be disposed within the lumen 104 of the guide catheter 102. This may include disposing the inflation lumen/tube 114 along the inner wall surface of the guide catheter 102. Alternatively, portions or all of the inflation lumen 114 may be radially spaced from the inner wall surface of the guide catheter 102.

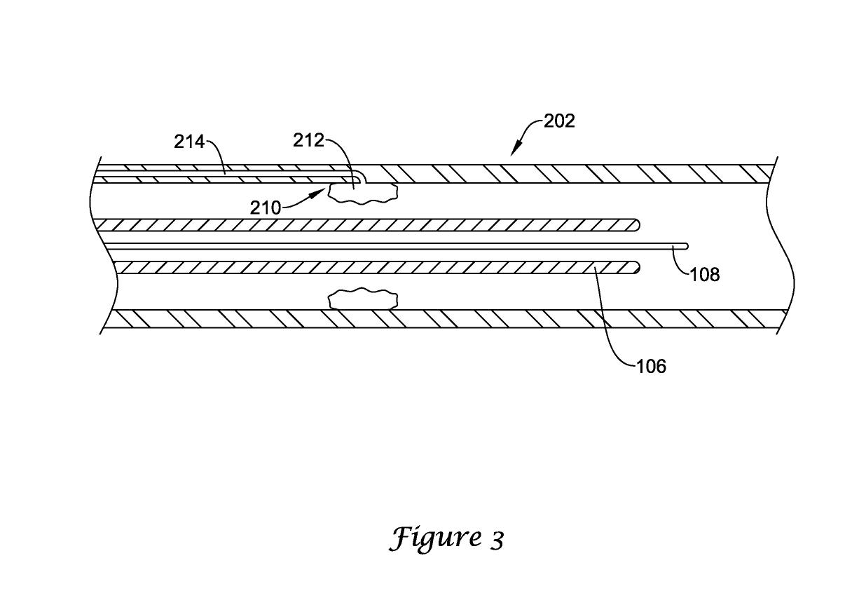

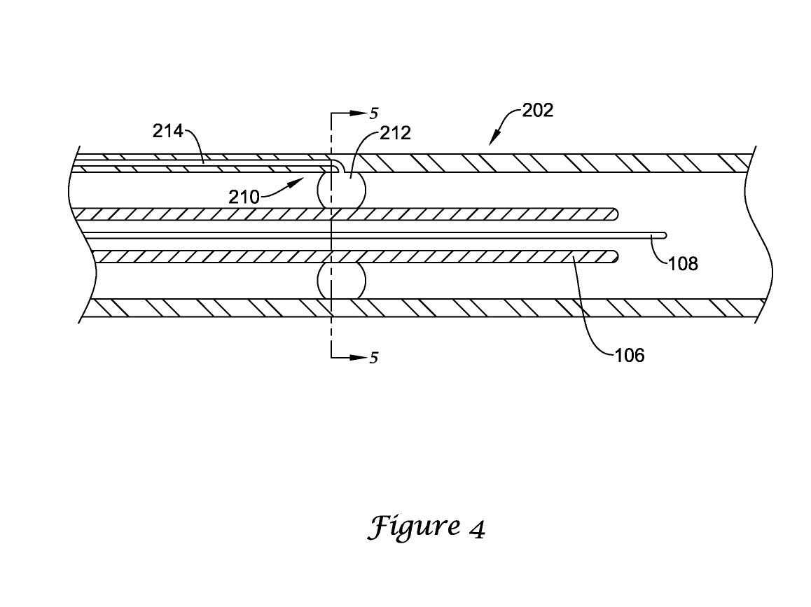

The inflation lumen 114 may be positioned in other locations. For example, FIG. 3 depicts another example guide catheter 202 that may be similar in form and function to other guide catheters disclosed herein. Guide catheter 202 may include a locking member 210 in which inflation lumen 214 passes through the wall of the guide catheter 202. This positioning of the inflation lumen 214 may increase the amount of space available for introducing medical devices within lumen 204. Locking member 210 may also include an inflatable balloon 212, which may be inflated to secure the position of the medical device 106 relative to the guide catheter 202 as shown in FIG. 4.

As discussed, upon inflation, the inflatable balloon 212 may expand into contact with the medical device 106, exerting a compressive force on it. This force may increase friction between the guide catheter 202 and the medical device 106, and if the compressive force is sufficiently strong, it will preclude relative motion between the two. As a result, the guide catheter 202 and the medical device 106, joined by balloon 212, become in effect a single unit, having increased stiffness as compared to either the guide catheter 202 or the medical device 106 individually. The increase in stiffness also may increase the “pushability” of the combined device. Increased stiffness may allow the guide catheter 202/medical device 106 to more efficiently cross a lesion or a torturous path in the patient's vasculature.

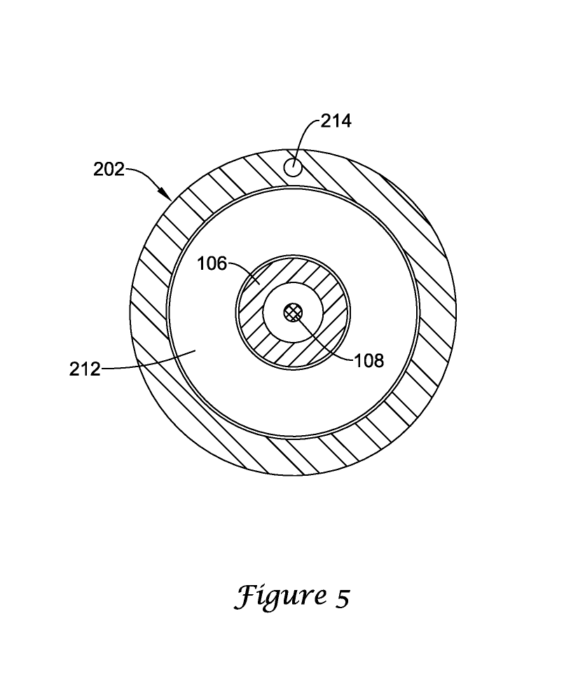

FIGS. 5-7 illustrate alternate configurations of the inflatable locking members and inflation lumens contemplated for any of the guide catheter disclosed herein. Referring to FIG. 5, in a configuration, the wall of the guide catheter 202 may include the inflation lumen 214, and the inflatable balloon 212 may have an annular shaped lobe. Upon inflation, the inflatable balloon 212 may grip the medical device 106.

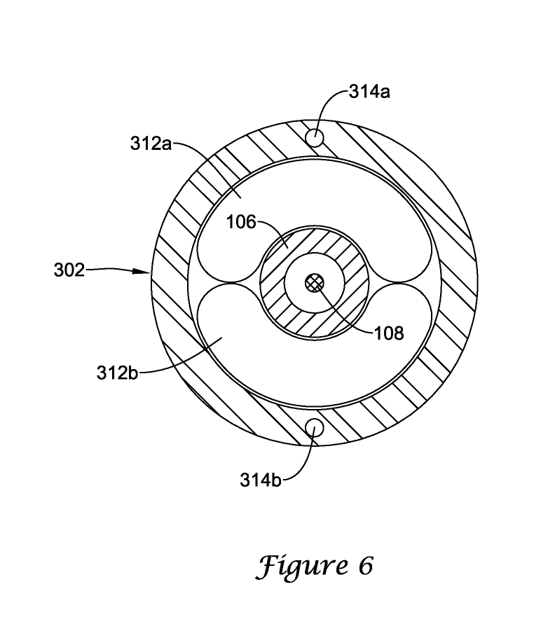

Alternatively, referring to FIG. 6, another example guide catheter 302 is shown that may include two inflation lumens 314a/314b positioned, for example, opposite each other within the wall of the guide catheter 302. Each inflation lumen 314a/314b may be in fluid communication with balloon sections or lobes 312a/312b, which may be semi-annular lobe in shape. Each lobe 312a/312b may be inflated/deflated independently.

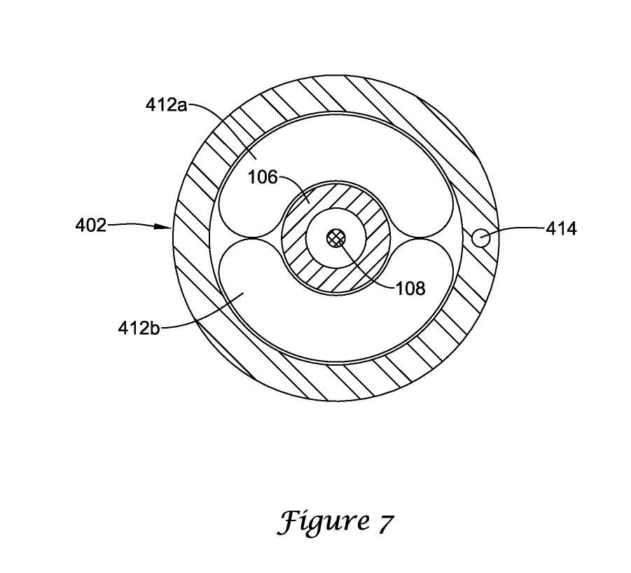

Further, referring to FIG. 7, yet another alternate configuration is shown where a single inflation lumen 414 extends through the wall of the guide catheter 402. The inflation lumen 414 may be in fluid communication with two semi-annular balloon lobes 412a/412b. The inflation lumen 414 may be positioned at a location adjacent to both the balloon lobes 412a/412b and may be used to simultaneously inflate both the balloon lobes 412a/412b.

These are just example configurations. Numerous other configurations are contemplated and may be utilized for any of the guide catheters disclosed herein.

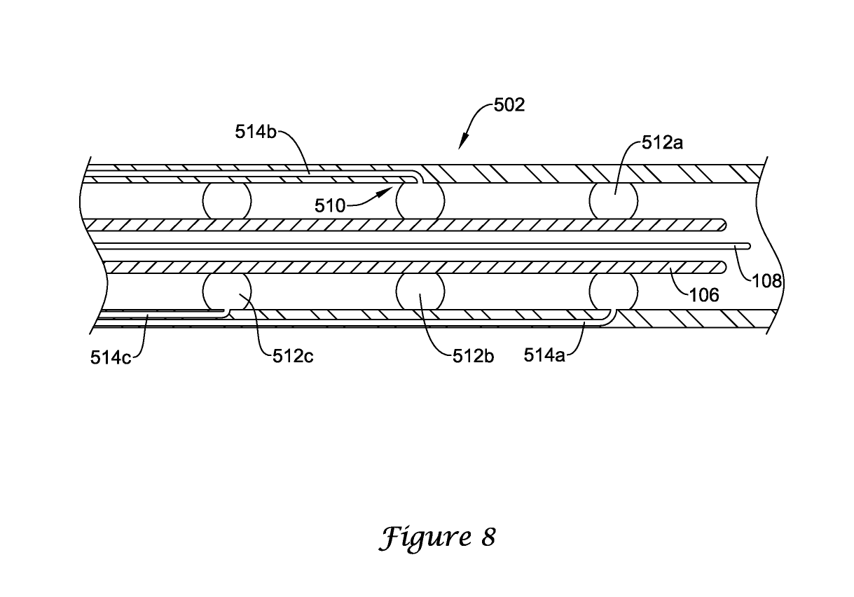

FIG. 8 depicts another example guide catheter 502 that may be similar in form and function to other guide catheters disclosed herein. In some embodiments, as shown, the guide catheter 502 may include the locking member 510 including a plurality of balloons 512a/512b/512c and a plurality of inflation lumens 514a/514b/514c (which may be in fluid communication with the balloons 512a/512b/512c, respectively). The inflatable locking members (e.g., balloons 512a/512b/512c) may be positioned at regular or irregular distances apart from each other along the length of the guide catheter 502. In at least some embodiments, each of the balloons 512a/512b/512c may be inflated independently of one another. This arrangement may be utilized to secure the medical device 106 relative to the guide catheter 502 and may improve pushability.

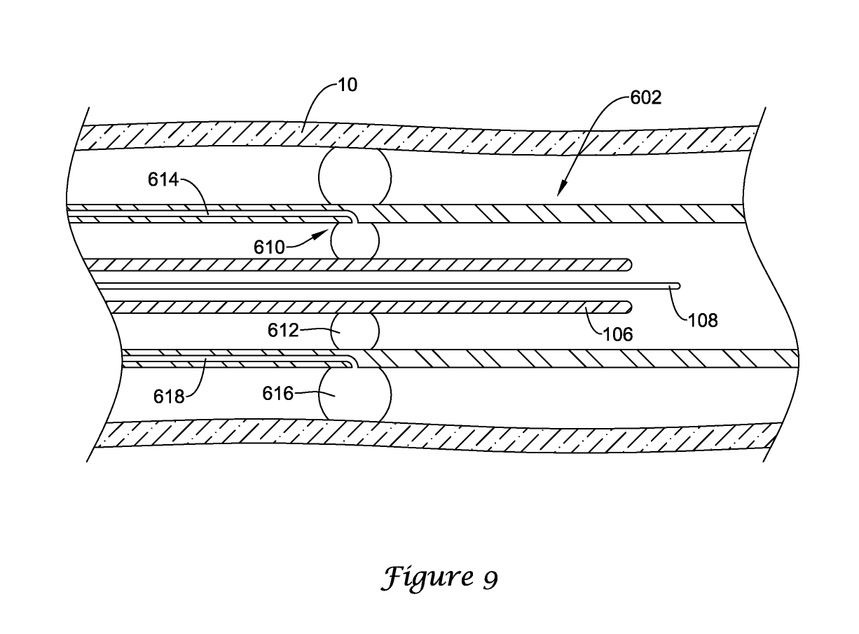

FIG. 9 illustrates another example guide catheter 602 that may be similar in form and function to other guide catheters disclosed herein. In some implementations, along with inflatable locking member 610 (e.g., including the balloon 612 and the inflation lumen 614), the guide catheter 602 may include exterior inflatable member 616. That member inflates to bear against the interior wall of the blood vessel 10, further supporting guide catheter 602 in a stable, stationary position. As shown, the exterior inflatable member 616 may be attached to the outer surface of the guide catheter 602. The exterior inflatable member 616 may be in fluid communication with the inflation lumen 614. Alternatively, balloon 616 may attach to additional inflation lumen 618, which extends through the wall of the guide catheter 602 (or, alternatively, along the inner surface of the guide catheter 602 or along the outer surface of the guide catheter 062). Upon expansion, exterior balloon 616 may firmly hold the guide catheter 602 in position, thereby allowing the guide catheter 602 to anchor at a desired position within the blood vessel 10.

A wide range of materials may be used to make the guide catheters disclosed herein (and/or the components thereof) including metals, polymers, metal-polymer composites, and the like. Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; combinations thereof; and the like; or any other suitable material.

Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), Marlex high-density polyethylene, Marlex low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenyleneterephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like. These are just examples.

In some implementations, composite materials may be employed. A suitable composite material may be a polymeric material reinforced with metallic wires braid or springs. Another suitable composite material may include short concentric polymeric and metallic tubes joined together in an alternating fashion to form the guide catheter and/or the components thereof. The polymeric portions may provide flexibility, while the metallic portions may add rigidity. Flexibility may allow the guide catheter to traverse circuitous paths, while stiffness may allow an operator to urge the guide catheter forward within the vasculature. To reduce friction, the outer surface of guide catheter may be coated with a suitable low-friction material, such as TEFLON®, polyetheretherketone (PEEK), polyimide, nylon, polyethylene, or other lubricious polymer coatings.

Although the embodiments described above have been set out in connection with a stent delivery system, those of skill in the art will understand that the principles set out here can be applied to any catheter or endoscopic device where it is deemed advantageous to limit relative motion between the guide catheter and a medical device extending through it. For example, the described guide catheter may be used in passing through tortuous paths in both cardiac and peripheral interventional devices, renal ablation devices, endoscopy and laparoscopy. Conversely, constructional details, including manufacturing techniques and materials, are well within the understanding of those of skill in the art and have not been set out in any detail here. These and other modifications and variations my well within the scope of the present disclosure and can be envisioned and implemented by those of skill in the art.

Other embodiments of the present disclosure will be apparent to those skilled in the art from consideration of the specification and practice of the embodiments disclosed herein. It is intended that the specification and examples be considered as exemplary only, and departure in form and detail may be made without departing from the scope and spirit of the present disclosure as described in the following claims.

What is claimed is:

1. A guide catheter, comprising:
an elongate catheter shaft having a proximal end, a distal end, and a central lumen extending therebetween;
an inflatable locking member disposed within the lumen, the locking member being configured to longitudinally secure the position of a medical device extending through the lumen relative to the catheter shaft; and
an inflation lumen in fluid communication with the inflatable locking member.

2. The guide catheter of claim 1, wherein the inflatable locking member includes an inflatable balloon.

3. The guide catheter of claim 2, wherein the inflatable balloon includes a single balloon lobe.

4. The guide catheter of claim 2, wherein the inflatable balloon includes a two or more balloon lobes.

5. The guide catheter of claim 1, wherein the inflation lumen is defined by a tubular member extending along an inner surface of the catheter shaft.

6. The guide catheter of claim 1, wherein the inflation lumen is defined within a wall of the catheter shaft.

7. The guide catheter of claim 1, further comprising one or more additional inflatable locking members disposed within the lumen.

8. The guide catheter of claim 1, further comprising a securing member for longitudinally securing the position of the guide catheter within a body lumen.

9. The guide catheter of claim 8, wherein the securing member includes an inflatable exterior balloon.

10. The guide catheter of claim 1, wherein the medical device includes a guidewire.

11. A guide catheter, comprising:
an elongate catheter shaft having a central lumen defined therein;
a balloon positioned within the lumen, the balloon being configured to shift between a first configuration and an inflated configuration;
an inflation lumen in fluid communication with balloon; and
wherein when the balloon is in the inflated configuration the balloon longitudinally secures the position of a medical device extending through the lumen relative to the catheter shaft.

12. The guide catheter of claim 11, wherein the balloon includes only a single balloon lobe.

13. The guide catheter of claim 11, wherein the balloon includes a two or more balloon lobes.

14. The guide catheter of claim 11, wherein the inflation lumen is defined by a tubular member extending along an inner surface of the catheter shaft.

15. The guide catheter of claim 11, wherein the inflation lumen is defined within a wall of the catheter shaft.

16. The guide catheter of claim 11, further comprising one or more additional balloons disposed within the lumen.

17. The guide catheter of claim 11, further comprising an exterior balloon disposed along an outer surface of the catheter shaft for longitudinally securing the position of the guide catheter within a body lumen.

18. A method for longitudinally securing medical devices, the method comprising:
disposing a first medical device within a body lumen, the first medical device comprising:
an elongate shaft having central lumen defined therein,
a balloon disposed within the lumen, and
an inflation lumen in fluid communication with the balloon;
disposing a second medical device within the central lumen of the first medical device; and
inflating the balloon to longitudinally secure the position of the second medical device relative to the first medical device.

19. The method of claim 18, wherein the first medical device includes a guide catheter and wherein the second medical device includes a guidewire.

20. The method of claim 18, wherein the first medical device includes an exterior balloon disposed along an outer surface of the shaft, and further comprising inflating the exterior balloon to longitudinally secure the position of the first medical device relative to the body lumen.
